(19) United States
(12) Patent Application Publication
(10) Pub. No.: US 2016/0113807 A1
(43) Pub. Date: Apr. 28, 2016

(54) Stability and Safety Weight Belt with Integrated Stability Straps and Safety Harness

(71) Applicant: David D. Hinton, SR., Moundville, AL (US)

(72) Inventor: David D. Hinton, SR., Moundville, AL (US)

(21) Appl. No.: 14/922,027

(22) Filed: Oct. 23, 2015

Related U.S. Application Data
(60) Provisional application No. 62/068,361, filed on Oct. 24, 2014.

(51) Int. Cl. A61F 5/02

(52) U.S. Cl. CPC A61F 5/028

(57) ABSTRACT

A weight belt having attachment loops located in both the right and left hip vicinity for use with a stability strap and/or a safety strap such that the stability strap attaches to the attachment loops and is placed around a piece of exercise equipment and such that the safety strap can be looped securely around a piece of exercise equipment and attached to the attachment loops. The weight belt also has apertures in the lower edge of the belt by which a safety harness can be attached and integrated into the weight belt.

### CROSS-REFERENCE TO RELATED APPLICATIONS

The instant application claims priority to U.S. Provisional Application Ser. No. 62/068,361 filed Oct. 24, 2014 the subject matter of which is incorporated by reference in its entirety.

### FEDERALLY SPONSORED RESEARCH

None

### SEQUENCE LISTING

NONE

### FIELD OF THE INVENTION

The present invention relates to a weight belt and integrated harness that provides a user safety, support and stability features while using exercise equipment.

### BACKGROUND OF THE INVENTION

Weight belts have been traditionally used to give abdominal and back support to an individual while performing exercises with or without weights. However, a common weight belt does not provide the stability and safety support that certain individuals require. Specifically, people who have suffered injuries in car accidents and other types of traumatic bodily injury have decreased motor skills and function of their stabilizer muscles than they once had. These individuals are often prescribed physical therapy treatment at exercise rehabilitation centers in order to regain their previous physical abilities. Due to their injuries, individuals in rehabilitation therapy are faced with the difficult task of lifting weights and performing exercise with an injured and weak body subject to falling, slipping, stumbling and becoming unstable during exercise. While traditional weight belts serve their purpose, there is a need in the art for a weight belt that can securedly couple a user to a piece of exercise equipment to keep a person stable while performing exercises on said equipment. A further need in the art is a weight belt that can prevent injury to a user while they are on a piece of exercise equipment such as a treadmill, an elliptical walking machine, a stair step exercise machine and other similar equipment. In accordance, a weight belt that provides stability and safety to a user while operating exercise equipment is needed in the art.

### SUMMARY OF THE INVENTION

The present invention is a weight belt that provides stability and safety to a user on various types of exercise equipment. The weight belt has a buckle and corresponding holes with which to secure the weight belt around a user for traditional use. Specifically, the invention is a weight belt of sufficient thickness in both height and width that has on its exterior attachment loops located in the area of the belt that corresponds to a user's right and left hip. In one embodiment, the weight belt has an exterior layer and an interior layer. The attachment loops can be made of metal, fabric, plastic or any other strong material that can support a user's weight under stress. The attachment loops can be attached to the exterior of the belt or to the interior of the belt by sewing, riveting, gluing or a combination of these methods. A stability strap with a first end and a second end is provided for use with the belt.

The stability strap has installed on each end a connection device such as a spring snap hook, a carabiner or other similar connection device that allows the stability strap to be connected to the weight belt via the attachment loops. The connection device is installed on the first and second ends of the stability strap by looping said strap around said connection device and sewing each respective end of the stability strap to itself. Additionally, a slide strap buckle is installed onto the stability strap to allow a user to cinch or expand the stability strap. The connection device on each end of said stability strap is intended to be connected to the attachment loops located on the weight belt.

In use, a user will buckle the weight belt around their waist. They will then proceed to loop the stability strap around the exercise equipment as the case may require. The stability strap can either be looped around a piece of equipment that a user is facing or it can be looped around a piece of equipment behind them. The user will then attach the connection devices on the stability strap to the respective attachment loops located on the exterior of the weight belt. Finally, the user will adjust the stability strap using the slide strap buckle located on the stability strap to ensure appropriate secure coupling of a user to said exercise equipment. The combination of abdominal and lower back support provided by the weight belt and stability provided by the stability strap ensures maximum comfort and safety to a user while exercising. Common exercise equipment suitable for use with the stability strap includes various weight benches that require a user to lie down thereon. In addition, the weight belt and stability strap are suited for various exercise machines that require a user to sit down upon a seat with a back rest or a chest rest.

It is a further object of the invention to have the weight belt provide upright safety to users operating equipment that requires them to walk, jog or move their legs in a walking fashion such as on treadmills, stair step machines, elliptical machines and other similar equipment. Specifically, a safety restraint is provided that allows a user to be attached to said exercise equipment to prevent or minimize the effects of a fall during operation of exercise equipment. The safety restraint has a loop on its tag end, a central length of strap extending from said loop and said length of strap converging into a junction where a left safety strap and a right safety strap extend a length beyond said junction. The left and right safety straps have attached on their ends a connection device such as a carabiner, spring snap hook or other similar rigid connection device. In addition, the left and right safety straps have attached along their length a slide strap buckle for adjustment of the length of the left and right safety straps.

Both the left safety strap and right safety strap are attached to the central length of strap. In one embodiment, the central length of strap is sewn onto the left and right safety straps. However, various forms of connection can be used including a three way junction plate whereby the central length and right and left safety straps are connected to a metal plate having three slots for each strap such that the straps are rendered to be in a Y formation. An additional method of connection is having the central length of strap and the right and left safety straps connected to a central metal loop. The central metal loop can be of various shapes such as circular, oval, rectangular, triangular and other geometric shapes.

In use, the loop on the tag end of the safety restraint is installed around a rigid portion of an exercise machine such that the left and right safety strap is threaded through said loop and pulled tight to form a secure connection to the exercise equipment. The connection devices located on the ends of the left and right safety straps are connected to the attachment loops on the weight belt. A user will use the slide strap buckles to adjust length of the left and right safety straps to a point where they feel that the restraint will hold their weight in an appropriate fashion should they stumble or fall.

It is further provided in an embodiment of the present invention to provide a safety harness integral with the weight belt. In accordance, the weight belt has integrated into its lower edge a plurality of holes that are fitted with grommets for reception and installation of a harness by use of a connection device such as a spring snap, carabiner or other similar device. The harness is constructed of cloth or fabric material and can be made out of a number of materials such as cotton, nylon, polyester, and other fibers. The harness has two inner straps and two outer straps. The two inner straps and the two outer straps merge into a rear support portion. A junction plate is provided to connect the two inner straps to rear slots formed through the junction plate. A front slot is provided in the junction plate for the two outer straps to pass through allowing them to be adjusted by the use of a drawstring lock slide or other similar device. The harness has in its rear support portion an aperture and a grommet installed therein for connection with the grommets in the weight belt. A spring snap hook, carabiner or other similar device is installed into the front slot of the junction plate and hooked into one the grommets located on the weight belt. The grommet found in the rear support portion is also connected to one of the grommets on the weight in like fashion.

In use, a user inserts their legs between the inner and outer straps so that the inner straps come into contact with the groin of a user and the outer straps come in contact with a user's hips. The user can then fasten the harness to the weight belt using the above described grommets and connection devices. The user can adjust the tension of the harness by pulling the tag ends of the two outer straps that are situated through the junction plate and by operating the drawstring lock slide to a desired fit.

### BRIEF DESCRIPTION OF THE DRAWINGS

The present invention can be best understood in connection with the accompanying drawings. It is noted that the invention is not limited to the precise embodiments shown in the drawings, in which:

FIG. 1. is a perspective view of the weight belt.

FIG. 2 is a perspective view of the weight belt and left attachment loop.

FIG. 3 is a perspective view of the stability strap

FIG. 4 is a perspective view of the safety restraint.

FIG. 5 is a top perspective view of the safety harness.

### DETAILED DESCRIPTION

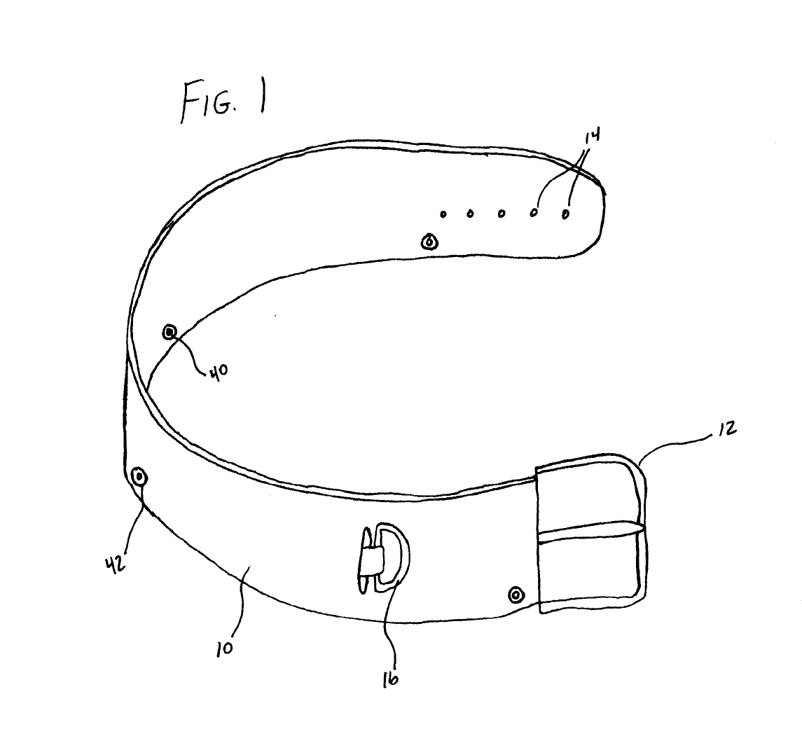

FIG. 1 shows a perspective view of the right side of the weight belt 10, a buckle 12, and holes for fastening the buckle. FIG. 1 further shows the left attachment loop 16 installed into the weight belt 10. The weight belt 10 can be made of leather, synthetic leather, nylon, polyester, polypropylene and other similar materials. In one embodiment, the weight belt 10 can be made of more than one layer that are fastened together by sewing, riveting, or gluing or a combination thereof. One of the layers can be foam padding placed in appropriate locations in the interior of the weight belt 10. Also depicted in FIG. 1 is the right attachment loop 16. The right attachment loop 16 can be made of metal, fabric, plastic or any other strong material that can support a user's weight under stress. The right attachment loop 16 can be installed either directly onto the exterior of the weight belt 10 or it can be installed into the interior of the weight belt 10 by method of sewing, gluing, riveting or a combination thereof. The right attachment loop 16 is installed at located on the exterior of the weight belt 10 that will generally align with a user's right hip. Further depicted in FIG. 1 are apertures 40 and grommets 42 on the lower edge of the weight belt 10.

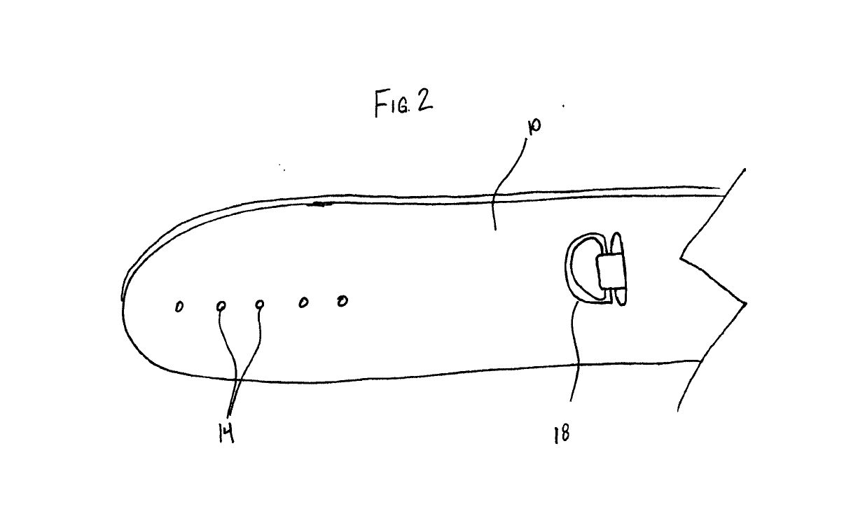

FIG. 2 shows the weight belt 10, holes 14 formed in one end of the weight belt 10 and the left attachment loop 18. The left attachment loop 18 can be installed onto said weight belt 10 in the same fashion as described above with regards to FIG. 1 as to the right attachment loop 16. The left attachment loop 18 is installed at located on the exterior of the weight belt 10 that will generally align with a user's right hip.

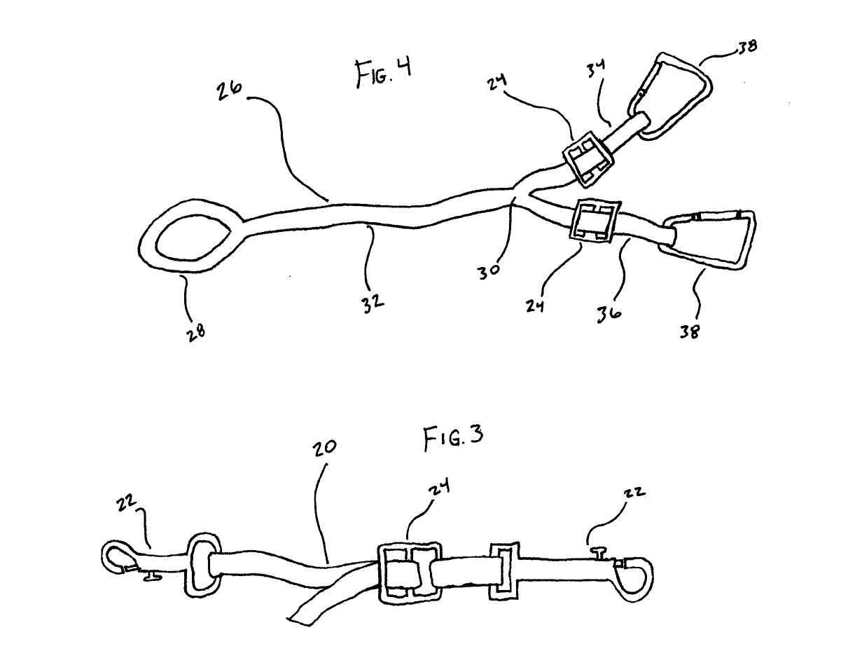

FIG. 3 depicts the stability strap 20 having a means for connecting 22 such as a spring snap hook, carabiner or other similar connecting device on each of the stability strap 20 two ends. The stability strap 20 can be made of nylon, polyester, polypropylene or other similar materials. While the stability strap 20 can be of many different lengths, a length of two to three feet is optimal for most situations. In addition, FIG. 3 depicts a slide strap buckle 24 which is used to cinch or expand the stability strap 20 to give a user the desired stability. In use, a user will buckle the weight belt 10 around their waist. They will then proceed to loop the stability trap 20 around the piece of exercise equipment they desire to use. The stability strap 20 can either be looped around a piece of equipment that a user is facing or it can be looped around a piece of equipment that is behind them depending on how the exercise equipment dictates a user's relative position. The user will then attach the connection device 22 onto the right attachment loop 16 and the left attachment loop 18 of the weight belt 10. Finally, the user will adjust the stability strap 20 using the slide strap buckle 24 located on the stability strap 20 to ensure appropriate secure coupling of a user to said exercise equipment.

FIG. 4 depicts a safety restraint 26 having a loop 28 at its tag end, a central length of restraint 32 and a junction 30. Further depicted is a left safety strap 34 and right safety strap 36 attached to said central length of restraint 32 at a junction 30. The left safety strap 34 and right safety strap 36 have slide strap buckles 24 installed upon their length for adjustment of the safety restraint 26. On the tag end of the left safety strap 34 and the right safety strap 36 are connection devices 38. The connection device 38 can be a spring snap hook, a carabiner or other similar devices that can provide a rigid connection and support the weight of a user. In one embodiment, the central length of restraint 32 is attached to the left safety strap 34 and right safety strap 36 by means of sewing. However, various other forms of junction 30 are possible such as a central rigid plate or loop that would allow the central length of restraint 32, the left safety strap 34 and the right safety strap 36 to all be connected thereto and thus allowing the Y shaped formation necessary for proper installation of the safety restraint 26 onto the weight belt 10.

In use, the loop 28 on the tag end of the safety restraint 26 is installed around a rigid portion of an exercise machine such that the left safety strap 34 and right safety strap 36 is threaded through said loop 28 and pulled tight to form a secure connection to the exercise equipment. The connection device 38 located on the ends of the left safety strap 34 and right safety strap 36 are connected to the right attachment loop 16 and left attachment loop 18 on the weight belt 10. A user will use the slide strap buckles 24 to adjust the length of the left safety strap 34 and right safety strap 36 to a point where they feel that the restraint will hold their weight in an appropriate fashion should they stumble or fall.

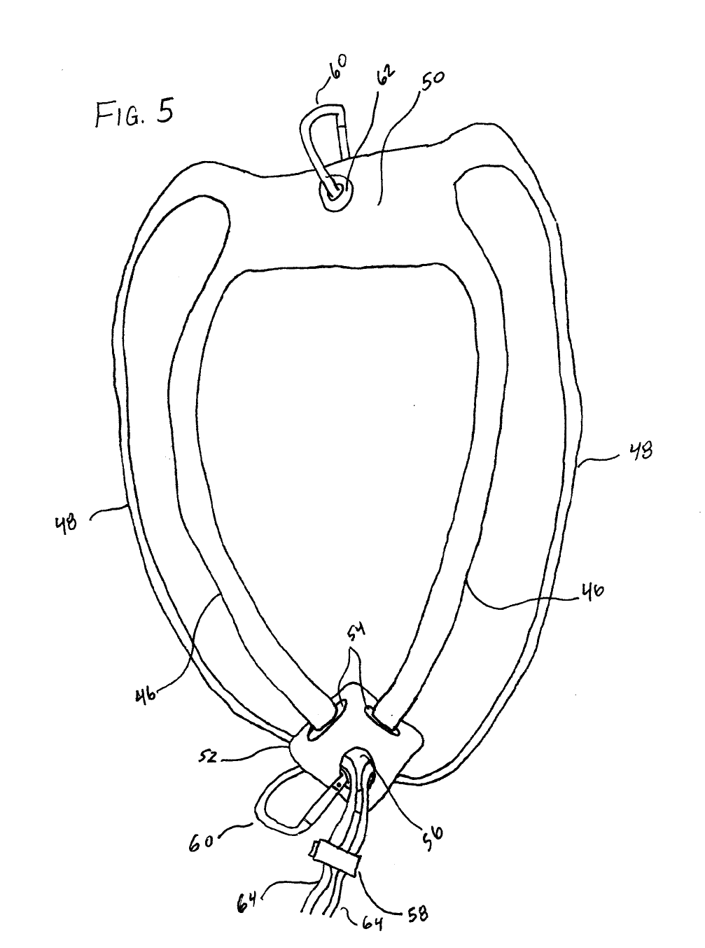

FIG. 5 shows a safety harness 44 that is designed for connection to the weight belt 10. In accordance, the weight belt 10 has integrated into its lower edge a plurality of apertures 40 that are fitted with grommets 42 (as shown in FIG. 1) for reception and installation of a harness 44 by use of a connection device 38 such as a spring snap, carabiner or other similar device. The harness 44 is constructed of cloth or fabric material and can be made out of a number of materials such as cotton, nylon, polyester, and other fibers. The safety harness 44 has two inner straps 46 and two outer straps 48. The two inner straps 46 and the two outer straps 48 merge into a rear support portion 50. A junction plate 52 is provided to connect the two inner straps 46 to the rear slots 54 formed through the junction plate 52. A front slot 56 is provided in the junction plate 52 for the two outer straps 48 to pass through allowing them to be adjusted by the use of a means for adjustment 58 such as a drawstring lock slide or other similar device. The harness 44 has a rear support portion aperture and grommet 62 installed therein for connection with the grommets 42 (as shown in FIG. 1) in the weight belt 10. A connection device 60 such as a spring snap hook, carabiner or other similar device is installed into the front slot 56 of the junction plate 52 and hooked into one the grommets 42 (as shown in FIG. 1) located on the weight belt. The rear support portion 50 aperture and grommet 62 found in the rear support portion 50 is also connected to one of the grommets 42 on the weight belt 10 (as shown in FIG. 1) by a connection means 60.

In use, a user inserts their legs between the inner straps 46 and outer straps 48 so that the inner straps 46 come into contact with the groin of a user and the outer straps 48 come in contact with a user's hips. The user can then fasten the harness 44 to the weight belt 10 (as shown in FIG. 1) using the above described grommets 42 (as shown in FIG. 1) and 62 and means for connection 60. The user can adjust the tension of the harness 44 by pulling the tag ends 64 of the two outer straps 48 that are situated through the junction plate 52 front slot 56 by operating the drawstring lock slide 58 to a desired fit.

I claim:

1. An exercise weight belt, comprising:
a weight belt having opposite first and second ends and a longitudinal axis extending between said ends;
a buckle attached to said first end;
holes formed in said second end for locking said buckle;
a left attachment loop located on the exterior of said weight belt and in the vicinity of a user's left hip;
a right attachment loop located on the exterior of said weight belt and in the vicinity of a user's right hip;
a stability strap adapted to allow a user to be securely coupled to a piece of exercise equipment, said stability strap has a stability strap first end and a stability strap second end, said stability strap first and second ends having means for connecting said stability strap first and second ends to said attachment loops on said weight belt; and
a slide strap buckle integrated into said stability strap.

2. The weight belt of claim 1, wherein the weight belt is formed of an inner layer and an exterior layer.

3. The weight belt of claim 1, wherein said attachment loops are fastened into the interior of said weight belt.

4. The weight belt of claim 1, further comprising slide strap buckles installed onto said left and right straps.

5. An exercise weight belt, comprising:
a weight belt having opposite first and second ends and a longitudinal axis extending between said ends;
a buckle attached to said first end;
holes formed in said second end for locking said buckle;
a left attachment loop located on the exterior of said weight belt and in the vicinity of a user's left hip;
a right attachment loop located on the exterior of said weight belt and in the vicinity of a user's right hip;
a stability strap adapted to allow a user to be securely coupled to a piece of exercise equipment, said stability strap has a stability strap first end and a stability strap second end, said stability strap first and second ends having means for connecting said stability strap first and second ends to said attachment loops on said weight belt;
a slide strap buckle integrated into said stability strap;
a safety restraint having a loop at its tag end, a central length of strap extending from said loop and said central length of strap converging into a junction where a left safety strap and a right safety strap extend a length beyond said junction and are adapted to be attached to a means for connection on the ends of said left and right safety straps for connection with said attachment loops; and
a slide strap buckle integrated into said left safety strap and a slide strap buckle integrated into said right safety straps.

6. The weight belt of claim 5, wherein the weight belt is formed of an inner layer and an exterior layer.

7. The weight belt of claim 5, wherein said attachment loops are fastened into the interior of said weight belt.

8. The weight belt of claim 5, further comprising slide strap buckles installed onto said left and right straps.

9. An exercise weight belt, comprising:
a weight belt having opposite first and second ends and a longitudinal axis extending between said ends;
a buckle attached to said first end;
holes formed in said second end for receiving said buckle;
a left attachment loop located on the exterior of said weight belt and in the vicinity of a user's left hip;
a right attachment loop located on the exterior of said weight belt and in the vicinity of a user's right hip;
a stability strap adapted to allow a user to be securely coupled to a piece of exercise equipment, said stability strap has a stability strap first end and a stability strap second end, said stability strap first and second ends having means for connecting said stability strap first and second ends to said attachment loops on said weight belt;
a slide strap buckle integrated into said stability strap;
a safety restraint having a loop at its tag end, a central length of strap extending from said loop and said central length of strap converging into a junction where a left safety strap and a right safety strap extend a length beyond said junction and are adapted to be attached to a means for connection on the ends of said left and right safety straps for connection with said attachment loops;
a slide strap buckle integrated into said left safety strap and a slide strap buckle integrated into said right safety straps;
a plurality of apertures formed in the lower edge of said weight belt;
a plurality of grommets installed into said apertures;
a harness comprising two inner straps, two outer straps, said two inner straps and said two outer straps merging into a rear support portion, and a junction plate;
said junction plate having three slots formed therein whereby two rear slots are formed in the rear portion of the junction plate and a front slot formed in the front of said junction plate;
said two inner straps fixedly secured to said rear slots of the junction plate;
said two outer straps fed through said front slot of said junction plate;
means for adjusting the tension of said two outer straps of said harness;
an aperture and grommet located in said rear central portion;
a connection device that attaches said weight belt to said central rear portion aperture and grommet; and
a connection device that attaches said junction plate front slot to said weight belt.

10. The weight belt of claim 9, wherein said weight belt is formed of an inner layer and an exterior layer.

11. The weight belt of claim 9, wherein said attachment loops are fastened into the interior of said weight belt.

12. The weight belt of claim 9, further comprising slide strap buckles installed onto said left and right straps.
